(19) United States
(12) Patent Application Publication
(10) Pub. No.: US 2013/0158420 A1
(43) Pub. Date: Jun. 20, 2013

(54) SYSTEM AND METHOD FOR ANALYSIS AND RECONSTRUCTION OF VARIABLE PULSE-WIDTH SIGNALS WITH FINITE-RATES-OF-INNOVATION

(71) Applicants: Roy Franklin Quick, JR., San Diego, CA (US); Ronald Eldon Crochiere, San Diego, CA (US); John Hyunchul Hong, San Clemente, CA (US)

(72) Inventors: Roy Franklin Quick, JR., San Diego, CA (US); Ronald Eldon Crochiere, San Diego, CA (US); John Hyunchul Hong, San Clemente, CA (US)

(73) Assignee: QUALCOMM Incorporated, San Diego, CA (US)

(21) Appl. No.: 13/552,206

(22) Filed: Jul. 18, 2012

Related U.S. Application Data
(60) Provisional application No. 61/570,741, filed on Dec. 14, 2011.

(51) Int. Cl. G06F 15/00; G06G 7/62; H04B 1/66; A61B 5/0402

(52) U.S. Cl. 600/509; 702/176; 703/13; 375/240

(57) ABSTRACT

Systems and methods are described herein for defining and parameterizing signals or system responses containing pulses of varying width. The parameters may define the signal and therefore can be equated to a compressed version of the original signal. Storage of the parameters as a compressed version of the signal requires less storage space, making storage of signals more memory efficient

### CROSS REFERENCE TO RELATED APPLICATIONS

This application claims priority under 35 U.S.C. Section 119(e) to U.S. Provisional Application 61/570,741, filed on Dec. 14, 2011.

### TECHNICAL FIELD

This disclosure relates to models for defining and parameterizing signals or system responses and a method for analyzing signals and system responses to determine these parameters.

### DESCRIPTION OF THE RELATED TECHNOLOGY

Signal parameterization is widely used in signal processing, storage, transmission, and analysis. Perhaps the most common is the use of Nyquist rate sampling, where a continuous time domain signal is represented by a set of sampled signal values at discrete times. As long as the original continuous signal is band limited to at most half the sampling rate, the set of samples can be used to reconstruct the complete signal by using, for example, a sinc interpolation algorithm. In this common example, the signal is represented by a set of discrete parameters, the sample values, which can be stored, transmitted, and used at any time to completely reconstruct the original signal.

More recently, some non-bandlimited signals of practical interest have been parameterized in other ways. Although these signals may contain frequency components that are arbitrarily large, they are modeled by characteristics that limit the “rate-of-innovation” per unit time so that the signal can be parameterized with a finite set of values from which the original signal can be reconstructed. The problem to be solved then is how to derive a suitable set of parameter values from the original signal and how to reverse the process to reconstruct the complete signal using only the derived parameters. The prior art of Finite Rate of Innovation (FRI) based signal analysis is one such method of analyzing signals and is described in the following references: [1] M. Vetterli, P. Marziliano, T. Blu, “Sampling Signals with Finite Rate of Innovation”, IEEE Transactions on Signal Procesing, vol. 50, no. 6, pp. 1417-1428, June 2002; [2] T. Blu, P. L. Dragotti, M. Vetterli, P. Marziliano, and L Coulot, “Sparse Sampling of Signal Innovations: Theory, Algorithms, and Performance Bounds”, IEEE Signal Processing Magazine, vol. 25, no. 2, pp. 31-40, March 2008; [3] Y. Hao, P. Marziliano, M. Vetterli, T. Blu, “Compression of ECG as a Signal with Finite Rate of Innovation”, Proc. of the 2005 IEEE Engineering in Medicine and Biology 27^{th} Annual Conference, Shanghai, China, Sep. 1-4, 2005, pp. 7564-7567; [4] Marziliano, M. Vetterli and T. Blu, “Sampling and Exact Reconstruction of Bandlimited Signals With Additive Shot Noise,” IEEE Transactions on Information Theory, vol. 52, No. 5, pp. 2230-2233, May 2006. In this method, waveforms or derivatives of waveforms are analyzed to identify the locations and amplitudes of Dirac functions (essentially infinitely narrow pulses) which are used to define signal features or markers for the boundaries of signal segments within the pseudo-periods. The signals or signal segments may then be reconstructed using the appropriate wave shapes or splines. These models (which may be referred to as Dirac-FRI models) described in the prior art are limited to two parameters for pulse descriptions, an amplitude and a position within a period (for pseudo-periodic signals), and they are restricted to a single pulse shape for reconstruction. Thus these models are limited in their ability to parameterize signals containing pulses of varying widths.

### SUMMARY

The systems, methods, and devices of the invention each have several aspects, no single one of which is solely responsible for its desirable attributes. Without limiting the scope of this invention as expressed by the claims which follow, some features will now be discussed briefly. After considering this discussion, and particularly after reading the section entitled “Detailed Description” one will understand how the features of this invention provide advantages that include models for defining and parameterizing signals or system responses containing pulses of varying width. The parameters may define the signal and therefore can be used as a compressed version of the original signal. Storage of the parameters as a compressed version of the signal requires less storage space, making storage of signals more memory efficient.

As provided below, a computer implemented method of compressing or decompressing a signal may include modeling the signal as a series of overlapping pulses having peak positions, damping factors, and amplitudes and performing the compression or decompression in accordance with the model.

In one implementation, a computer implemented method of signal parameterization includes obtaining, by at least one processor, a series of at least MM discrete Fourier transform coefficients of an N sample time domain signal, where MM is greater than 2K, and determining, by the at least one processor, K roots of an annihilator polynomial using the MM discrete Fourier transform coefficients. Based at least in part on the determined roots, a location and width of K pulses is derived by the at least one processor. A real or complex amplitude for each of the K pulses is also derived by the at least one processor.

In another implementation, a non-transient computer readable media having instructions stored thereon can cause processing circuitry to perform the steps of obtaining a series of MM discrete Fourier transform coefficients, determining K roots of an annihilator polynomial using the MM discrete Fourier transform coefficients, and based at least in part on the determined roots, deriving the locations, the widths, and the real or complex amplitudes of each of K pulses.

In another implementation, an apparatus configured for signal parameterization includes a processor configured to obtain a series of frequency domain transform coefficients of a time domain signal, determine roots of an annihilator polynomial using the frequency domain transform coefficients, and based at least in part on the determined roots, derive a location and width of pulses of the time domain signal. Such an apparatus may be coupled to ECG electrodes as part of an ECG monitoring system.

In another implementation, an apparatus configured for signal parameterization includes means for generating a time domain signal and means for compressing the time domain signal by deriving a location and a width of pulses in the time domain signal from frequency domain coefficients of the time domain signal.

### BRIEF DESCRIPTION OF THE DRAWINGS

FIGS. 1A-1C illustrate reconstruction of an ECG waveform with a sum of Lorentzian pulses.

FIG. 2 is a flow chart of a method of parameterizing waveform containing variable width pulses.

FIG. 3 is a graph of the spectrum of an ECG waveform.

FIG. 4 is a flowchart of a method of signal reconstruction using the parameters generated with the method of FIG. 2.

FIG. 5 is a graph comparing an original time domain signal with a reconstruction of the signal using VPW-FRI parameters.

FIG. 6 is a graph comparing the time domain waveforms of FIG. 5 in the frequency domain.

FIGS. 7A-7C illustrates embodiments of systems utilizing signal parameterization of the present disclosure.

FIG. 8 illustrates an embodiment of a system utilizing signal parameterization of the present disclosure.

### DETAILED DESCRIPTION

Various aspects of the novel systems, apparatuses, and methods are described more fully hereinafter with reference to the accompanying drawings. The teachings may be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the invention. For example, an apparatus may be implemented or a method may be practiced using any number of the aspects set forth herein. In addition, the scope of the invention is intended to cover such an apparatus or method which is practiced using other structure, functionality, or structure and functionality in addition to or other than the various aspects of the invention set forth herein. It should be understood that any aspect disclosed herein may be embodied by one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different systems, some of which are illustrated by way of example in the figures and in the following description of the preferred aspects. The detailed description and drawings are merely illustrative of the disclosure rather than limiting, the scope of the disclosure being defined by the appended claims and equivalents thereof

The methods of the present disclosure may be applicable to a variety of systems. For example, the present disclosure may be particularly applicable to signal storage databases such as electro-cardiogram (ECG) databases. In one aspect, the methods herein may be used to parameterize ECG signals and then store those parameters in an ECG database. This may greatly reduce the cost and resources required for storing ECG signals as the memory allocation required to store the data is greatly reduced. Such ECG signal databases provide information used for evaluation and treatment of patients. Further, hospitals may require such ECG databases as part of storage of patient medical records. Thus, the systems and methods described herein may prove valuable to the medical field.

Many of the methods described herein overcome difficulties of the prior art by introducing parameters in the model which define the widths and asymmetries of the pulse shapes of the model and extends the mathematics and methodology of FRI analysis. Additionally, the disclosure defines the means for performing the analysis to estimate these parameters from the data. It generalizes the model of FRI to a four-parameter pulse model which can represent waveforms as the sum of overlapping pulses, each of which is characterized by a position, symmetric component amplitude, asymmetric component amplitude, and width. The added degrees of freedom provided by this generalization greatly extend the range and class of signals that can be modeled and it permits the representation of overlapping of pulses rather than concatenations of waveform segments.

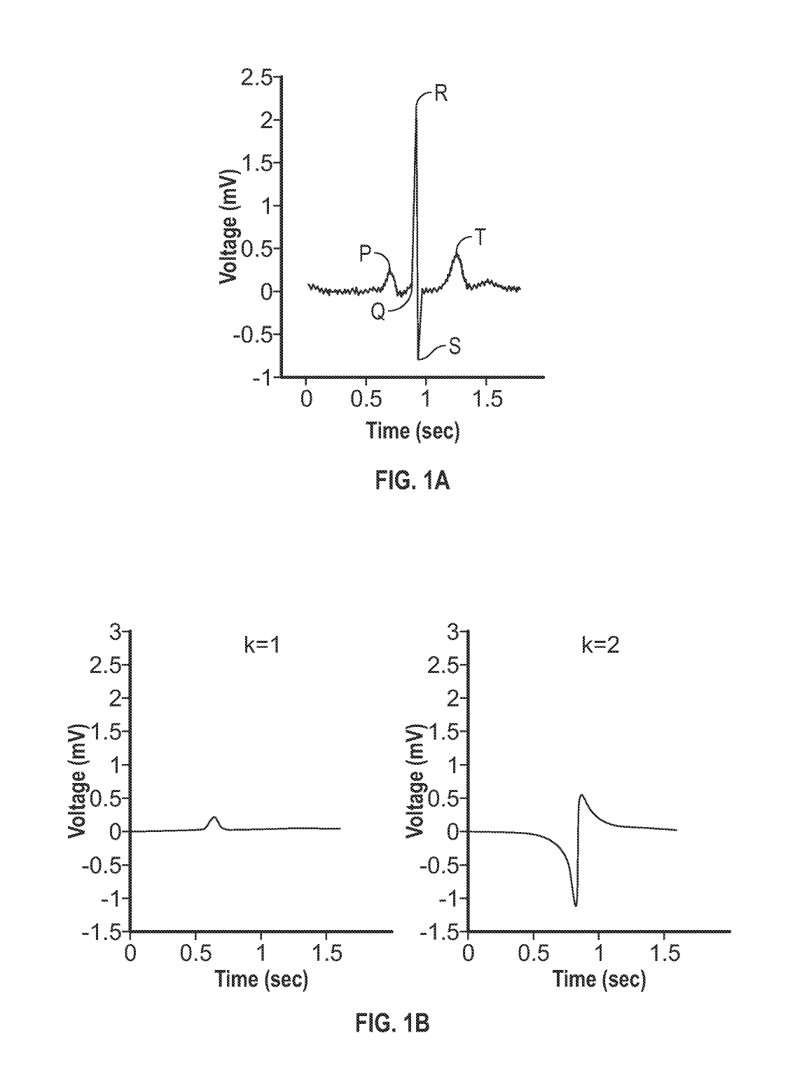

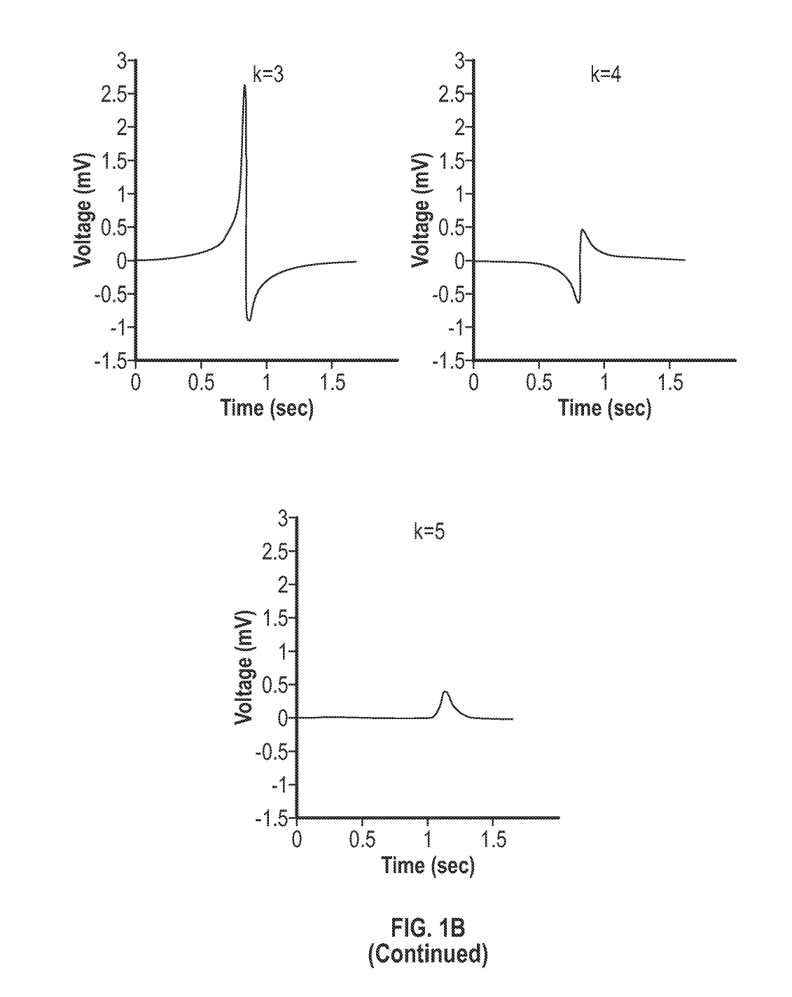

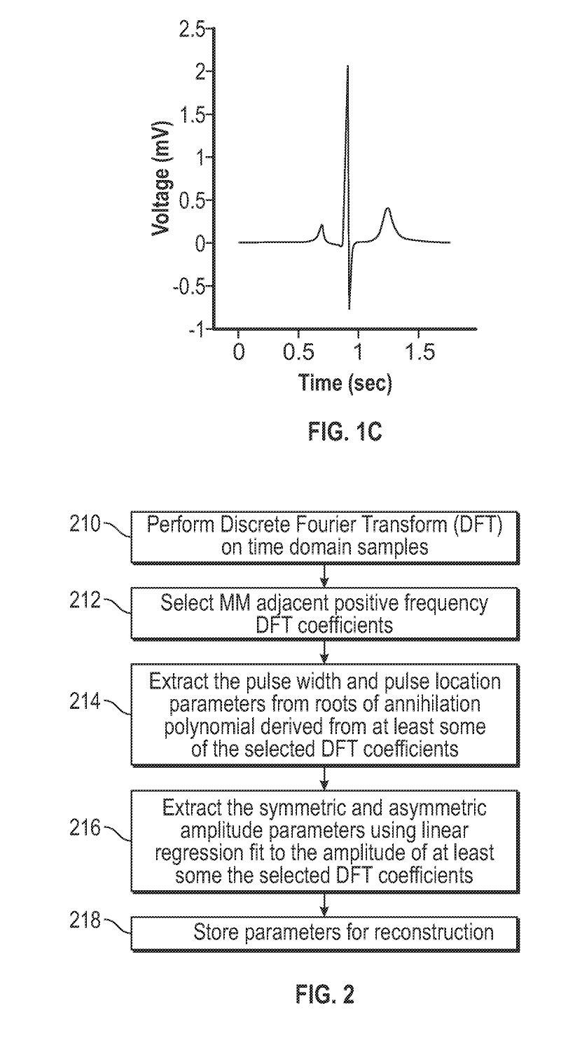

Signals that approximate the parameterization model described herein may be referred to as Variable Pulse-Width Finite Rate of Information (i.e. the VPW-FRI) signals. An example of this class of signals is the heart beat in an ECG waveform as illustrated in FIG. 1A. As seen by this figure, the structure of a heart beat is defined by its P Q R S T components. The location, size, and shape of these components convey critical information about the physical operation of the heart. In the VPW-FRI model, the waveform may be modeled as a sum of Lorentzian (also known as Cauchy distribution) pulses, each of which includes a symmetric and an asymmetric component:

$$x ( t ) = {\sum }_{k = 1}^{K} \frac{1}{π} \frac{{c}_{k} {a}_{k} + {d}_{k} ( t - {t}_{k} )}{{a}_{k}^{2} + {( t - {t}_{k} )}^{2}} \qquad ( 1 )$$

In this model, the waveform is considered to be formed from K pulses, each one denoted by an index k, and each one of which is defined by a center position tₖ, a width or damping factor aₖ, an amplitude for a symmetric pulse component cₖ, and an amplitude for an asymmetric pulse component dₖ. The number of Lorentzian pulses used to model a signal will depend on the nature of the signal. For an ECG waveform, using a K of five has been found generally suitable to reproduce the P, Q, R, S, and T features that are of clinical significance, although more robust results have been found in some cases if a six or seven pulse model is used, especially when the ECG waveforms being modeled have a relatively large amount of noise.

For some signal waveforms, the asymmetric amplitude dₖ can be set to zero, which reduces the number of parameters used to model the waveform. This is possible with the ECG waveforms, but the results in general are less accurate.

FIG. 1B illustrates five Lorentzian pulses of the form set forth in Equation 1 whose parameters were extracted from the original waveform of FIG. 1A using the methods described below. The sum of the pulses in FIG. 1B is shown in FIG. 1C. It can be seen that FIG. 1C is a good reproduction of the original signal of FIG. 1A. In this way, the signal from FIG. 1A is accurately parameterized by 20 parameters, four parameters (c, d, a, and t) for each of the five Lorentzian pulses of FIG. 1B and Equation 1.

As with other parameterization techniques, a method is required to derive the desired parameters from the original time domain data, which is typically a series of discrete waveform samples taken during the acquisition of an analog electrical signal output from electrodes coupled to a subject. The sampling rate may vary, but may be about 120-360 Hz, producing about 100-500 time domain samples of the waveform over the approximately 0.75 to 1.5 second time period of interest containing the P, Q, R, S, and T waveform features. It will be appreciated that parameterizing the waveform to 20 parameter values instead of the 100-500 original waveform sample values can produce a compression of the data by a factor of 5-25.

Although it may be possible to perform a derivation of the pulse parameters in the time domain (by, for example, using a regression analysis to find a, t, c, and d values for each of the five pulses that minimizes the differences between the values produced by Equation 1 at the sample times and the actual sample values), significant computational and accuracy advantages arise from performing the parameter derivation in the frequency domain.

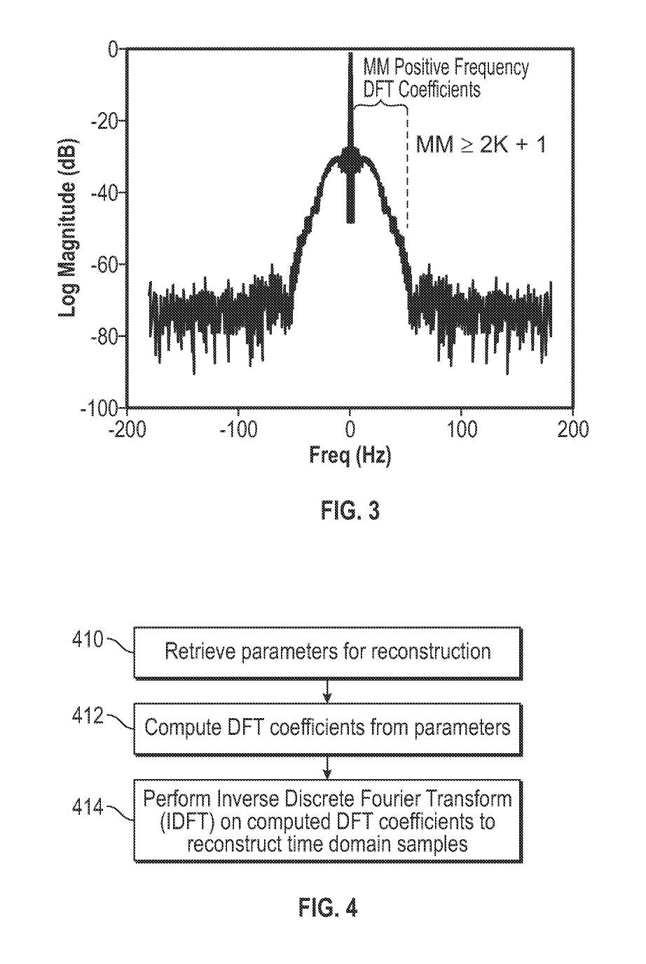

FIG. 2 is a flow diagram of one method of signal analysis for deriving the VPW-FRI parameters in the frequency domain. In this implementation, the method starts at block 210, where a discrete Fourier transform (DFT) is performed on the original time domain samples. The method continues at block 212, where a set of MM positive frequency DFT coefficients are selected for further analysis to extract the above described pulse parameters. These steps are also illustrated graphically in FIG. 3, which shows an ECG frequency spectrum produced from a DFT algorithm applied to a time domain ECG waveform sampled at 360 Hz. As can be seen in FIG. 3, such a spectrum typically includes a region of decaying oscillations around 0 Hz and a transition region where the spectral energy decreases with increasing frequency to 60 or 70 Hz. A noise floor of around −70 dB can also be seen in FIG. 3 at frequencies above about 70 Hz. The details of the ECG waveform affect the frequencies and decay rate in the oscillating region, and the details of the shape of the transition region.

The set of MM DFT coefficients selected for analysis is on the positive side of 0 Hz, and includes at least 2K+1 adjacent DFT coefficient values. For the extraction method described in more detail below, positive frequency coefficients are selected because the set of coefficients cannot span across 0 Hz due to the methods used to perform the extraction as described further below. The extraction method described below also requires at least 2K+1 values as inputs, although stability and accuracy of results in the presence of noise is improved if more than this number are utilized. If K=5 (e.g. 5 pulse model) for an ECG waveform, it has been found useful for the number MM to be at least 25 or 30 DFT coefficients rather than the minimum of 11. The selected set of coefficients should include the oscillating region from near 0 Hz and extend to cover at least some of the transition region as well.

Referring back to FIG. 2, at block 214, the pulse width and pulse location parameters aₖ and tₖ are extracted from the roots of an annihilation polynomial derived from at least some of the selected DFT coefficients. To extract the damping factor (also referred to as the pulse width factor) aₖ and the time tₖ of each pulse, the method assumes that the DFT of block 210 was generated from a sampled time domain signal having the functional form of Equation 1. The DFT coefficients of a sampled sum of such Lorentzian pulses will follow the form of a sum of decaying sinusoids as shown in Equation 2:

$$\hat{X} ( m ) = {\sum }_{k = 1}^{K} {b}_{k} {e}^{- ( 2 π / τ ) {a}_{k} \vert m \vert - j ( 2 π / τ ) {t}_{k} m} \\ for \\ m = - \frac{N}{2} … , 0 , … \frac{N}{2} - 1 \qquad ( 2 )$$

where bₖ=cₖ−jdₖ for positive m
bₖcₖ+jdₖ for negative m
τ=N/Fₛ, where Fₛ is the time domain sampling frequency and N is the number of time domain samples taken during the period of interest

If the DFT of the original signal is assumed to be of the functional form of Equation 2, an annihilating polynomial can be constructed having roots related to the parameters aₖ and tₖ. Techniques for solving this mathematical problem of deriving the parameters of a sampled (and possibly noisy) signal, where the signal is a sum of exponentially damped oscillations, have been developed and are well known. For example, spectral analysis techniques such as the Prony algorithm are known that can construct the annihilating polynomial and find its roots. Other spectral analysis techniques can also be used to find the roots of the annihilation polynomial such as ESPRIT. Such techniques have been used for similar purposes such as the Dirac-FRI methods mentioned above. Past attempts to use such methods, however, generally used DFT coefficients that include both positive and negative frequencies. When the pulses are assumed to have variable width, however, this presents a discontinuity, as can be seen from Equation 2, and the Prony algorithm, for example, does not model curves with exponential weights that change within the domain of analysis. One method of solving this problem is by recognizing that the spectrum of the time domain signal is conjugate symmetric, and it is not necessary that the coefficients used in the algorithm span 0 Hz. One can use only positive or only negative components of the spectrum (plus 0 Hz if desired) to satisfy the conditions of the Prony method. The complex roots of the annihilator polynomial in the spectral analysis will then provide the information to determine the damping factors in the model in addition to the pulse times. An additional advantage is gained by the recognition that frequency components supplied to the Prony method do not have to start at m=0. Since data obtained from real applications is often corrupted by DC offsets, it can be advantageous to avoid the m=0 term and, in some cases, the m=1 term in the analysis. The net effect is to make the analysis more robust to DC offsets which may occur in real data.

When one of the above described spectral analysis techniques is applied in this manner, this polynomial will have K roots which are (in polar form):

$${z}_{k} = {e}^{- ( 2 π / τ ) {a}_{k}} {e}^{j ( 2 π / τ ) {t}_{k}} \\ k = 1 , … K \qquad ( 3 )$$

It can be seen that the magnitude and phase of the roots are as follows:

$$\vert {z}_{k} \vert = {e}^{- ( 2 π / τ ) {a}_{k}} \\ ∠ {z}_{k} = \frac{2 π}{τ} {t}_{k} \qquad ( 4 )$$

From these roots, the pulse locations tₖ can be computed:

tₖ=(τ/2π)·∠zₖ for K=1, . . . , K (5)

where tₖ are the pulse locations in samples (0 to N−1) and the angle of zₖ is in radians (0 to 2π).

The damping factors, aₖ , are related to the magnitudes of the roots by the relation:

aₖ=−(τ/2π)ln|zₖ| (6)

After finding the damping factors and pulse times, at block 216 of FIG. 2 the symmetric and asymmetric amplitude parameters are extracted using a linear regression fit of DFT coefficients to be generated by Equation 2 to the DFT coefficients generated from the original time domain signal at block 210.

To accomplish this, a set of linear equations may be defined by matching a set of L values of X(m) from the input data with values expressed by the model in Equation 2. This set may be some or all of the MM previously selected coefficients, or any other selection of L values for m≧0. A minimum of K signal values and K equations (L≧K) are needed to form a matrix equation which may be inverted (or least squares inverted) to obtain the values of bₖ. In practice, more signal values are generally used and the values of bₖ are determined by a least squares method.

The above set of linear equations can be described as follows:

$$\hat{X} \{ m \} = {\sum }_{k = 1}^{K} {b}_{k} {e}^{- ( 2 π / τ ) {a}_{k} m - j ( 2 π / τ ) {t}_{k} m} = {\sum }_{k = 1}^{K} {b}_{k} {z}_{k}^{- m} m \geq 0 \\ where \text{:} \\ {z}_{k} = {e}^{( 2 π / τ ) {a}_{k} + j ( 2 π / τ ) {t}_{k}} , {b}_{k} = {c}_{k} - j {d}_{k} \qquad ( 7 a )$$

Defining:

G=└zₖ^{-m}┘(L×K matrix) (7b)

b=[bₖ](K×l column vector) (7c)

x=[X {m}](L×lcolumn vector)m={set of L freq samples} (7d)

The solution for c can be expressed as:

Gb=x (7e)

b=inv(G)x (7f)

where

inv(G) is the least squares (e.g. Moore-Penrose) pseudo-inverse of the matrix G.

When the components of the column vector [b] are determined with this method, the real part of each bₖ is the symmetric amplitude cₖ of pulse k, and the imaginary part of each bₖ is the asymmetric amplitude dₖ of pulse k.

After all the parameters aₖ, tₖ, cₖ, and dₖ are calculated, the parameters are stored at block 218 for future signal reconstruction. When this model is applied, the complete waveform can be compressed to four values for each pulse (plus potentially an additional DC shift parameter), which for a five pulse waveform is only twenty or twenty-one values, much less than the number of time domain (or frequency domain) values that would be stored as representative of the waveform if a conventional Nyquist rate sampling method were used.

FIG. 4 is a flow diagram of signal reconstruction from the stored parameters. In this implementation, the stored parameters aₖ, tₖ, cₖ, and dₖ are retrieved at block 410. From the stored parameters, at block 412 DFT coefficients for positive m frequency indices are computed using Equation 2 and the retrieved parameters. Coefficients for negative m frequency indices may be computed by taking the complex conjugates of the positive m values produced by Equation 2. At block 414, an inverse discrete Fourier transform (IDFT) is performed on the DFT coefficients, producing a set of time domain values that represent the original time domain signal. It will be appreciated that an alternative reconstruction method may be used in the time domain, where the parameters aₖ, tₖ, cₖ, and dₖ are plugged into Equation 1 to directly produce time domain data. Care should be taken in this case to use a shifted periodic version of Equation 1 if the original signal has any significant DC offset. This complication is not present if the frequency domain reconstruction of FIG. 4 is utilized.

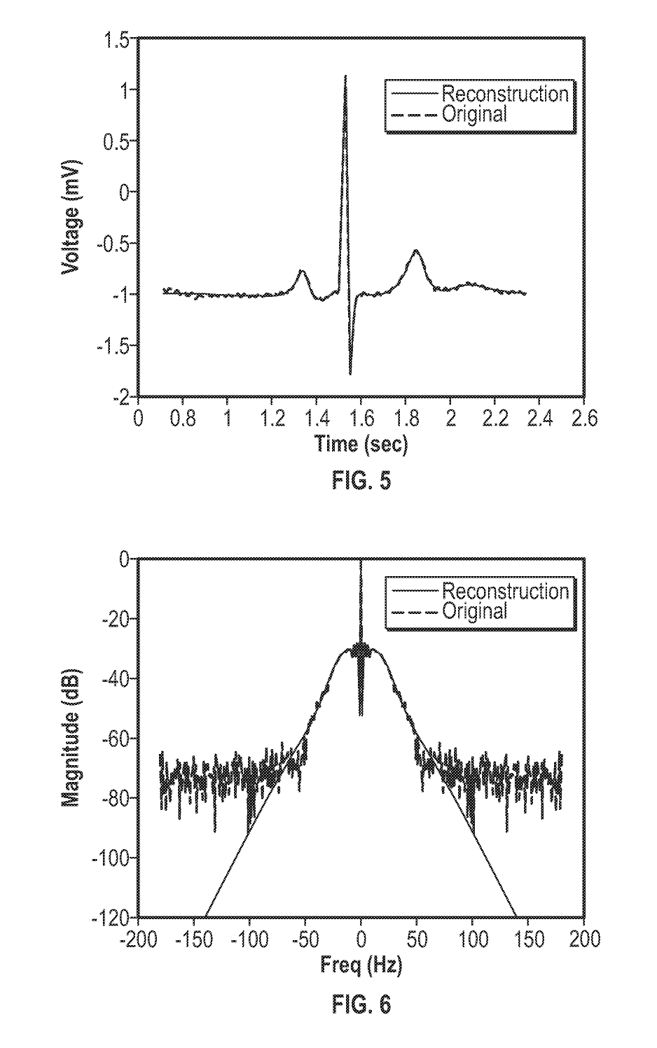

To illustrate the utility of this approach, the VPW-FRI model and the analysis method defined by this disclosure, FIGS. 5 and 6 show the results of the analysis of a real heart beat waveform obtained from the MIT/BIH data base. In Figure 5, the original time domain data is shown in dashed line, and the reconstruction output from the above described method (at block 414 of FIG. 4 for example) is shown in solid line. In FIG. 6, the DFT of the original signal (originally sampled at 360 Hz) is shown in dashed line, and the DFT coefficients produced by the method using Equation 2 at block 412 of FIG. 4 are shown in solid line.

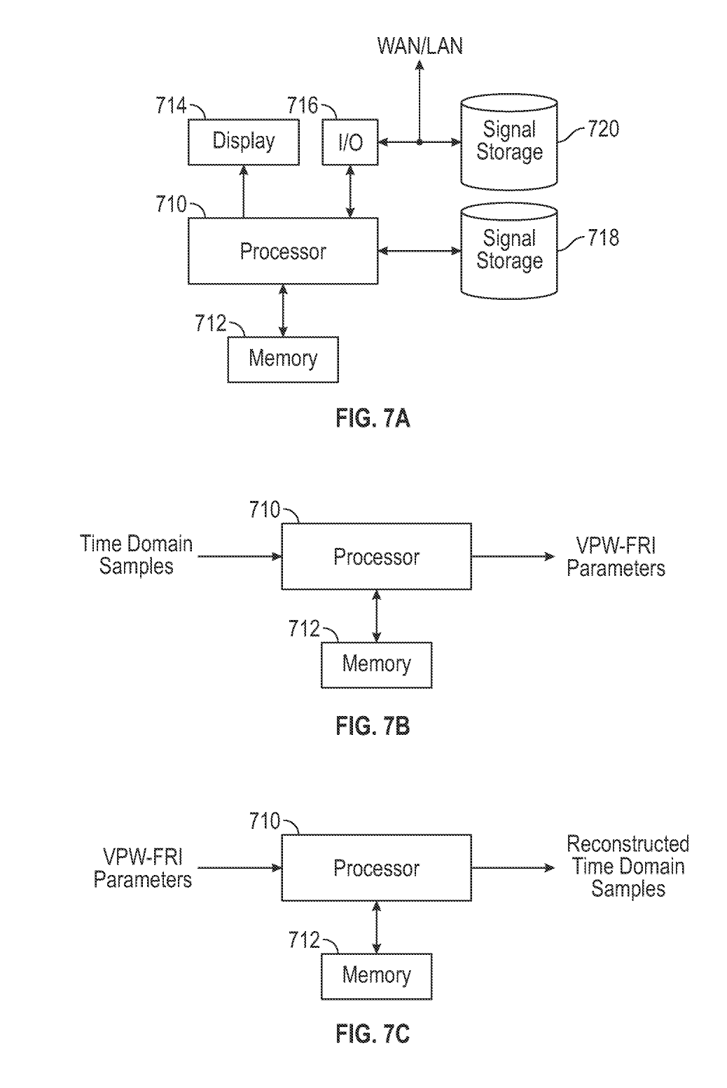

The methods described above can be implemented in a wide variety of systems. FIG. 7A illustrates a block diagram of one device configured to implement the VPW-FRI methods. The device may include a processor 710. The processor 710 may also be referred to as a central processing unit (CPU). Memory 712, which may include both read-only memory (ROM) and random access memory (RAM), provides instructions and data to the processor 710. A portion of the memory 712 may also include non-volatile random access memory (NVRAM). The processor 710 typically performs logical and arithmetic operations based on program instructions stored within the memory 712. The instructions in the memory 712 may be executable to implement the methods of the VPW-FRI model described herein. For example, the instructions in the memory 712 may be executable by the processor 710 to implement the sequence of signal processing steps for the analysis and/or reconstruction of a waveform as shown in FIGS. 2, 4, and 11.

The processor 710 may be configured to receive N samples (where N is a positive integer) of an input signal in the time domain from a local signal storage 718 or a remote signal storage 720. Further, the processor 710 may transform the N samples utilizing a DFT algorithm to produce the transform coefficients X(m) in the frequency domain as described above. Alternatively, the signal storage may store DFT coefficients of original time domain signals, and these could be used as a starting point for the algorithm implemented by the processor 710. A subset MM of the transform coefficients (whether received by or generated by the processor 710), in particular a set of coefficients associated with positive frequencies, may be selected by the processor 710. The subset MM of the transform coefficients may then be used by the processor to define the VPW-FRI parameters. These parameters may then be stored as compressed versions of the original data in signal storage 718 or 720. The original data can then be discarded or stored elsewhere. The processor may also be configured to retrieve VPW-FRI parameters from the signal storage memory 718 or 720. The processor may then generate DFT coefficients using the methods described above, and perform an IDFT on the generated DFT coefficients to reconstruct a time domain waveform. An output device such display 714 or a printer may be used to display either original or reconstructed time domain data.

The signal storage memory 718 and/or 720 may comprise an ECG signal database. The processor may then use as input ECG signals, and store them as parameters computed utilizing the VPW-FRI algorithm. Storage of the ECG signals as such parameters may reduce the memory allocation necessary for storing such ECG signals. The system of FIG. 7A may be employed, for example, at a hospital to efficiently store ECG signals as part of patient medical history records.

Although a number of separate components are illustrated in FIG. 7A, those of skill in the art will recognize that one or more of the components may be combined or commonly implemented. Further, each of the components illustrated in FIG. 7A may be implemented using a plurality of separate elements.

FIGS. 7B and 7C are block diagrams illustrating that different processors 710 which may be geographically separated can separately perform the signal analysis and signal reconstruction. In FIG. 7B, the processor 710 is dedicated to performing the signal analysis portion of the process. This processor takes time domain samples and produces VPW-FRI parameters. In FIG. 7C, the processor is dedicated to reconstruction. This processor takes VPW-FRI parameters as an input, and produces reconstructed time domain samples as an output.

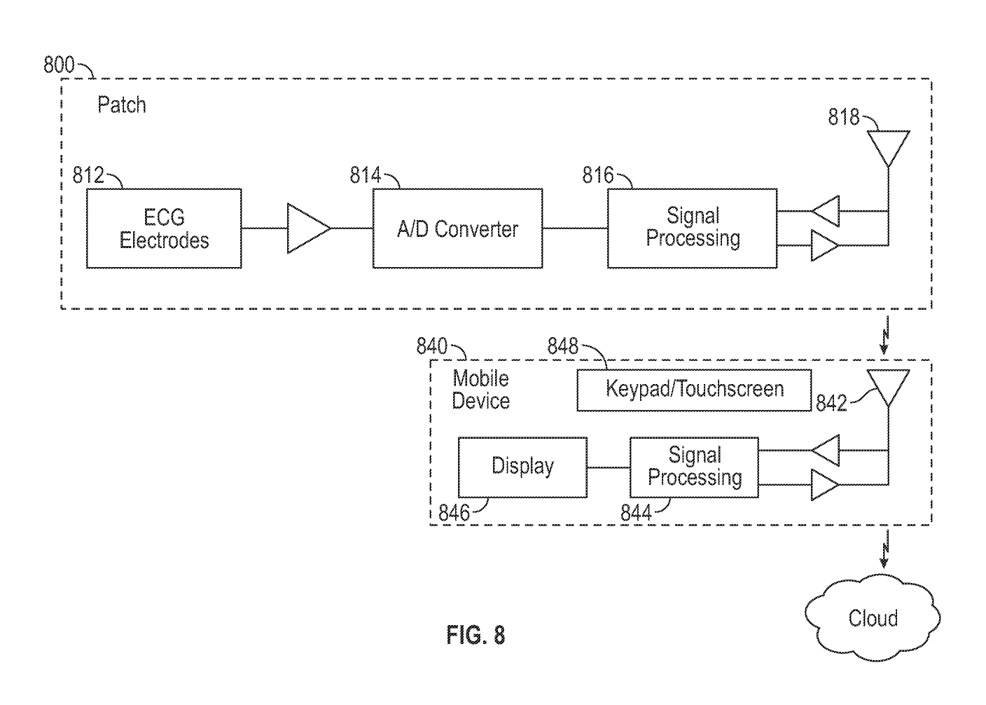

FIG. 8 illustrates another system in which the above described methods can be implemented. In this system, a patch ECG monitor 800 incorporates ECG electrodes 812 and is mounted with adhesive for example on a subject as an ambulatory cardiac monitoring device. The signal from the electrodes is routed to an A/D converter 814 which produces time domain samples of the signal. These samples are sent to signal processing circuitry 816 which may be configured to produce the VPW-FRI parameters of pulse width, time, and symmetric and asymmetric amplitude described above. These may be sent wirelessly via antenna 818 to a mobile device 840 such as a cell phone, tablet, or other portable electronic system, which receives the parameters via antenna 842 and routes the parameters to signal processing circuitry 844 in the mobile device 840. It will be appreciated that the components of the patch 800 need not be mounted together on the same physical substrate, but could be split up in a variety of ways.

The signal processing circuitry 844 in the mobile device 840 may be configured to reconstruct the ECG waveforms using the VPW-FRI parameters. The reconstructed signal may be displayed on a display 846 and manipulated with a keypad/touchscreen 848 on the mobile device. The mobile device may also be configured to transmit either the reconstructed waveform and/or the VPW-FRI parameters to an external network such as the Internet for storage, review by a physician, etc.

Because the on-body mounted system 800 should use as little power as possible, it is advantageous to minimize the sampling rate of the A/D converter and also minimize the amount of data that must be transmitted from the on-body system 800 to the mobile device 840. The compression and accurate reconstruction provided by the methods described above can reduce the power consumed by the on-body system 800.

The VPW-FRI method described above is based on pulse shapes (e.g. Lorentzian) whose spectra can be defined as exponentially damped sine waves with independent damping factors for each pulse (which is mathematically consistent with the Prony analysis). If the pulse spectra of a signal have different shapes, such as Gaussian shapes, then there will be a model mismatch between the inherent nature of the data and the model used by VPW-FRI. The effects of this mismatch can be partially reduced by multiplying the data by a pre-emphasis factor before using the VPW-FRI model and by a corresponding de-emphasis factor in the reconstruction method. The pre-emphasis can be applied to data X(m) of the input DFT by multiplying it by a pre-emphasis factor P(m)

The de-emphasis can similarily be applied to the reconstructed signal by dividing \hat{X}(m) by P(m) prior to taking the IDFT.

The choice of the pre-emphasis function P(m) would be determined by the application and, in its simplest form, it would be a static model.

The various illustrative logic, logical blocks, modules, and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a computer-readable medium. Computer-readable media includes both computer storage media and communication media including any medium that can be enabled to transfer a computer program from one place to another. A storage media may be any available media that may be accessed by a computer. By way of example, and not limitation, such computer-readable media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and Blu-Ray™ disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. A computer implemented method of signal parameterization, the method comprising:
obtaining, by at least one processor, a series of at least MM discrete Fourier transform coefficients of an N sample time domain signal, where MM is greater than 2 K;
determining, by the at least one processor, K roots of an annihilator polynomial using the MM discrete Fourier transform coefficients;
based at least in part on the determined roots, deriving, by the at least one processor, a location and width of K pulses; and
deriving, by the at least one processor, a real or complex amplitude for each of the K pulses.

2. The method of claim 1, wherein the pulses have an approximately Lorentzian function shape in the time domain.

3. The method of claim 1, wherein the MM discrete Fourier transform coefficients all correspond to frequencies greater than or equal to zero.

4. The method of claim 1, wherein the MM discrete Fourier transform coefficients all correspond to frequencies less than or equal to zero.

5. The method of claim 1, comprising deriving the amplitudes for the symmetric and asymmetric components of the K pulses.

6. The method of claim 1, wherein the amplitudes are determined by a linear regression matrix inversion.

7. The method of claim 1, comprising deriving the series of at least MM discrete Fourier transform coefficients from a series of time domain samples of the signal.

8. The method of claim 1, wherein the time domain signal comprises an electrocardiography (ECG) signal.

9. A non-transient computer readable media having instructions stored thereon causing processing circuitry to perform the steps of:
obtaining a series of MM discrete Fourier transform coefficients;
determining K roots of an annihilator polynomial using the MM discrete Fourier transform coefficients;
based at least in part on the determined roots, deriving the locations, the widths, and the real or complex amplitudes of each of K pulses.

10. A computer implemented method of compressing or decompressing a signal comprising modeling the signal as a series of overlapping pulses having peak positions, damping factors, and amplitudes, and performing the compression or decompression with a processing circuit in accordance with the model.

11. The method of claim 10, wherein the pulses have an antisymmetric component amplitude.

12. The method of claim 10, wherein the compressing comprises:
obtaining a series of at least MM discrete Fourier transform coefficients;
determining K roots of an annihilator polynomial using the at least MM discrete Fourier transform coefficients;
based at least in part on the determined roots, deriving a location, a width, and a real or complex amplitude of each of K pulses.

13. An apparatus configured for signal parameterization, the apparatus comprising:
a processor configured to:
obtain a series of frequency domain transform coefficients of a time domain signal;
determine roots of an annihilator polynomial using the frequency domain transform coefficients; and
based at least in part on the determined roots, derive a location and width of pulses of the time domain signal.

14. The apparatus of claim 13, wherein the processor is further configured to derive a real or complex amplitude for each of the pulses.

15. The apparatus of claim 13, wherein the pulses have an approximately Lorentzian function shape in the time domain.

16. The apparatus of claim 13, wherein the frequency domain transform coefficients all correspond to frequencies greater than or equal to zero.

17. The apparatus of claim 13, wherein the MM discrete Fourier transform coefficients all correspond to frequencies less than or equal to zero.

18. The apparatus of claim 13, wherein the processor is further configured to derive the amplitudes for symmetric and aymmetric components of the pulses.

19. The apparatus of claim 13, wherein the processor is further configured to derive the series frequency domain transform coefficients from a series of time domain samples of the signal.

20. The apparatus of claim 19, wherein the time domain signal comprises an electrocardiography (ECG) signal.

21. The apparatus of claim 19, wherein the apparatus comprises ECG electrodes, an AID converter, the processor, and an antenna configured for mounting on the body of a subject.

22. The apparatus of claim 21, wherein the apparatus comprises a portable device having an antenna and a processor, wherein the portable device is configured for communication with the processor.

23. An apparatus configured for signal parameterization, the apparatus comprising:
means for generating a time domain signal;
means for compressing the time domain signal by deriving a location and a width of pulses in the time domain signal from frequency domain coefficients of the time domain signal.

24. The apparatus of claim 23, wherein the means for generating a time domain signal includes electrodes.

25. The apparatus of claim 23, including means for transmitting the compressed time domain signal.
